(19) United States
(12) Patent Application Publication
(10) Pub. No.: US 2012/0083863 A1
(43) Pub. Date: Apr. 5, 2012

(54) Thermal brassiere push up

(76) Inventor: Kimberly Gwen Gillespie, Port Charlotte, FL (US)

(21) Appl. No.: 12/923,660

(22) Filed: Oct. 1, 2010

(51) Int. Cl. A61F 7/00; A41C 3/10; A41C 3/00

(52) U.S. Cl. 607/108; 607/114; 450/38; 450/89; 450/56

(57) ABSTRACT

A pouch to be placed into a brassiere and in other garment locations. The pouch consists of several layers of cotton fabric and neoprene fabric materials. At least one layer of neoprene fabric creates the base layer of the pouch. At least two layers of a cotton fabric material overlaps with neoprene fabric in a location which is about three-quarters from the edge of the pouch to form an entrance into said pouch for the placing of a thermal gel pack therein. The neoprene fabric layer is for the insulation of the thermal gel pack from the environment. The thermal gel pack inside the pouch, creates the thermal brassiere push up, which when worn in a brassiere cup provides a lift of the breast, enhances the woman's cleavage and provides concealed thermal comfort.

### BACKGROUND OF THE INVENTION

Brassiere push ups know to enhance the appearance of the female breasts. Other push ups known to provide a therapeutic treatment to the female breasts and the other parts of the human body.

U.S. Pat. No. 6,544,100 discloses a push up bra which has an envelope between the panel of the rear of the brassiere cup to receive a flexible pouch containing a mixture of water and a hydroscopic agents such as 70% glycerin-30% water. The hydroscopic agent will draw moisture into the envelope preventing the volume of liquid from decreasing.

U.S. Pat. No. 6,916,334 teaches the use of a thermal pack for the application of heat or cold to the breast. The pack comprises a pliable disk-shaped body having an aperture approximately through the center of the body. The body is constructed with a top wall having an outer surface and an inner surface and a bottom wall wherein the two surfaces define at least one fluid tight cavity and a thermal fluid cell, such as a gel, is contained within the cavity.

U.S. Pat. No. 7,309,275 discloses a brassiere incorporating at least one gel pack positioned to provide a thermal therapy to a breast. It is located within the brassiere cup. The gel pack is located within the breast cup construction. The gel bag forms an integral part of the garment and is sandwiched between layers of material forming the interior and exterior of the garment.

### BRIEF DESCRIPTION OF THE INVENTION

The present invention relates to a thermal brassiere push up, preferably cold, but not limited thereto. A thermal gel pack is placed within a neoprene fabric and cotton fabric pouch which when placed and worn under the breasts inside a brassiere cup becomes a thermal brassiere push up. It has been considered to be advantageous to provide a thermal brassiere push up worn under the breast within a brassiere cup for the purpose of providing concealed relief to the discomfort of environmental heat and temperature changes caused by hormones and physical activity .

It would also be advantageous to provide a thermal brassiere push up worn under the breasts and placed in the cups of a sports bra, racer back, type bra and an additional thermal gel pack in a pocket located between the shoulder blades on the back of the bra to provide concealed relief around the body.

It would also be advantageous to provide a thermal brassiere push up in the a single pocket inside a shelf bra, in which one thermal brassiere push up provides concealed relief to both breasts simultaneously.

It would also be advantageous to provide a thermal brassiere push up worn under the breast within a brassiere cup to lift and enhance the cleavage of the breasts.

### BRIEF DESCRIPTION OF THE DRAWINGS

FIG. 1 is a front plane view of pouch that contains the thermal gel pack;

FIG. 2 is a rear plane view showing the entrance to a pocket;

FIG. 3 is a cross section view with a thermal gel pack placed therein;

FIG. 4 is a cross section view with a thermal gel pack placed therein;

FIG. 5 is a front plane view of a brassiere depicting the placement of the thermal brassiere push up as worn.

### DETAILED DESCRIPTION OF THE INVENTION

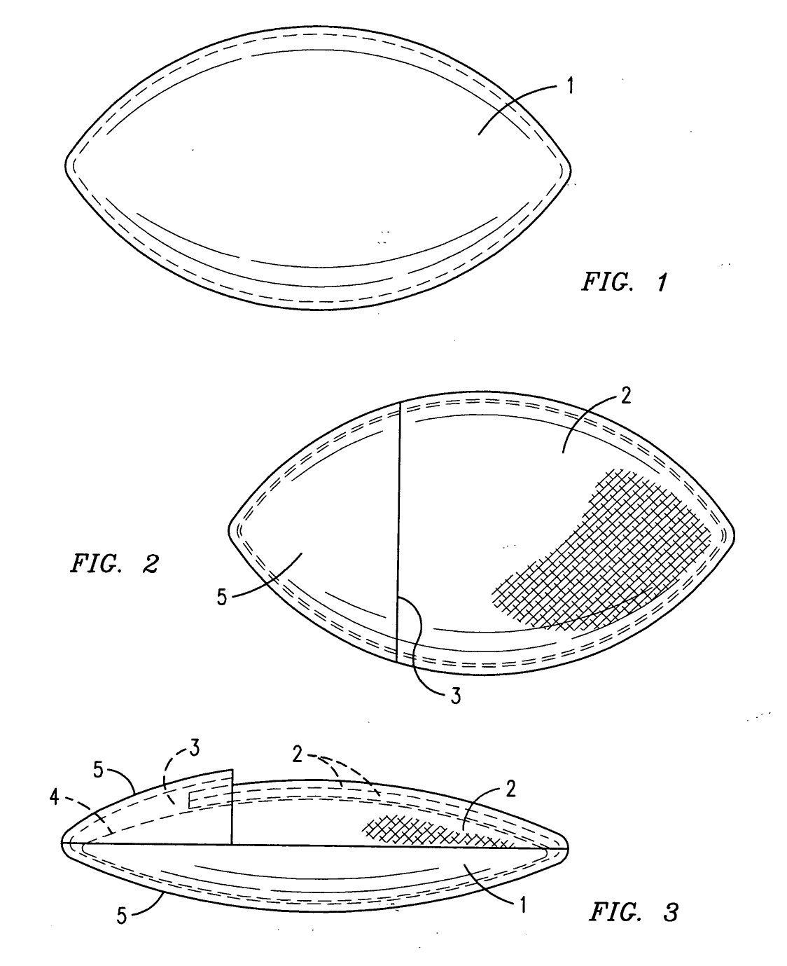

In FIG. 1 and in accordance with the present invention, there is shown a pouch 1 in front plan view designed to be placed within a brassiere, (FIG. 5). The pouch is preferably made and constructed in an elliptical shape so that it will conform to the inside of a brassiere and conform to the shape of a female breast. Of course, any other shape may be chosen for the application to different garment shapes and different parts of the human body.

FIG. 2 shows the shape of the pouch of FIG. 1 but in a rear view. The pouch is constructed as a pocket having a cotton fabric 2 and a neoprene fabric 5 on the rear side which is worn against the breast. There are multiple cotton fabric 2 layers coming from an edge of the pouch 1 and covering three-fourths of the whole extent of the pouch 1. There is one neoprene fabric 5 layer coming from the opposite edge of the pouch 1 and covering one-third of the whole extent of the pouch 1 to thereby create an overlap at 3 which also forms an entrance into the interior of the pouch.

FIG. 3 is a cross section view of the pouch 1 which has the thermal gel pack 4 placed under two cotton fabric 2 layers covering three-fourths of the extent the pouch 1 and on top of one cotton fabric 2 layer covering three-fourths of the extent the pouch 1 and under one neoprene fabric 5 layer coming from the opposite edge of the pouch 1 and covering one-third of the whole extent of the pouch 1 to thereby create an overlap at 3. This overlap provides an entrance into the pouch 1 as shown in FIG. 2. The thermal gel pack 4 is placed therein. The multiple cotton fabric layers provides an optional number of barrier layers. A layer of neoprene fabric 5 is the whole extent of the bottom layer of the pouch 1.

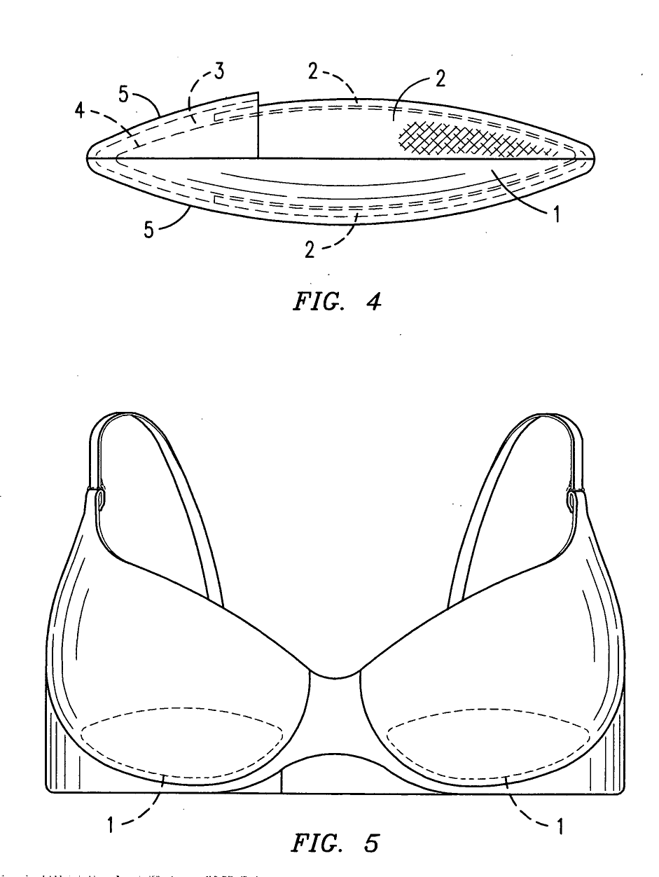

FIG. 4 is a top side cross section view of the pouch 1 showing the gel pack 4 placed under one cotton fabric 2 layer and on top of one cotton fabric 2 layer. The multiple cotton fabric layers 2 and the neoprene fabric 5 are shown providing the overlap and the entrance into the pouch 1 at 3. The thermal gel pack 4 is placed within. A layer of neoprene fabric 5 is the whole extent of the bottom layer of the pouch 1. The neoprene fabric layer 5 is instrumental for relieving the discomfort a body experiences from temperatures by increasing the usage time of the enclosed thermal gel pack. The gel in the thermal brassiere push up is a compound made of water, sodium polyacrylate (absorbent material) and propylene glycol such as 72% water, 5% sodium polyacrylate, and 23% propylene glycol. The portions of which are adjusted for form and volume.

FIG. 5 is a plane view of a brassiere depicting the placement of the pouch 1 as worn.

What I claim is:

1. A pouch constructed of multiple cotton fabric and neoprene fabric layers and designed to receive a thermal gel pack therein. A neoprene fabric layer creates the base layer of the pouch. Multiple cotton fabric layers overlap to the extent of about three quarters from the side edge of said pouch with a neoprene fabric layer which extends from the opposite edge of said pouch to the extent of one-third of the whole to form an entrance in the said pouch. A thermal gel pack is placed into said pouch.

2. The pouch of claim 1, wherein the thermal gel pack can be placed between any of the multiple cotton fabric layers.

3. The pouch of claim 1, wherein said pouch is worn concealed within a brassiere.

4. The pouch of claim 1, wherein the content of said thermal gel pack is a gel material.

5. The pouch of claim 1, wherein said gel material is a compound comprising of a mixture of water, sodium polyacrylate, and propylene glycol such as 72% water, 5% sodium polyacrylate, and 23% propylene glycol.
